(19) United States
(12) Patent Application Publication
(10) Pub. No.: US 2018/0021410 A1
(43) Pub. Date: Jan. 25, 2018

(54) METHODS FOR INCREASING PERFORMANCE CHARACTERISTICS IN OFFSPRING

(71) Applicant: Puretein Bioscience LLC, St. Louis Park, MN (US)

(72) Inventors: Brett Casebolt, Edina, MN (US); Robert Eldon Musser, Good Thunder, MN (US); Chad D. Hagen, Sleepy Eye, MN (US)

(21) Appl. No.: 15/547,926

(22) PCT Filed: Feb. 4, 2016
(86) PCT No.: PCT/US2016/016539

§ 371 Date: Aug. 1, 2017

Related U.S. Application Data
(60) Provisional application No. 62/111,975, filed on Feb. 4, 2015.

(51) Int. Cl. A61K 38/30; A23K 10/24; A23K 10/20; A23K 50/30

(52) U.S. Cl. CPC A61K 38/30; A23K 50/30; A23K 10/24; A23K 10/20

(57) ABSTRACT

Provided herein are methods for altering a characteristic of an offspring or a litter. The method can include administering an effective amount of a composition that includes active IGF-1 to a pregnant animal, wherein a characteristic of an offspring of the pregnant animal is altered compared to a control offspring. In another embodiment, the animal is a lactating animal, wherein a characteristic of an offspring nursed by the lactating animal is altered compared to a control offspring. Examples of characteristics that can be altered include increased survival of offspring before weaning, increased weight of offspring at weaning, increased weight of offspring at end of growing phase, increased average daily gain, and increased average daily food intake. In another embodiment, a characteristic of a litter can be altered, such as increased number of offspring born alive, increased litter birth weight, increased offspring birth weight, reduced number of stillborn offspring, or a combination thereof.

### CROSS-REFERENCE TO RELATED APPLICATIONS

This application claims the benefit of U.S. Provisional Application Ser. No. 62/111,975, filed Feb. 4, 2015, which is incorporated by reference herein.

### SUMMARY OF THE APPLICATION

Provided herein are methods for altering a characteristic of an offspring or a litter. In one embodiment, the method includes administering an effective amount of a composition that includes active IGF-1 to a pregnant animal, wherein a characteristic of an offspring of the pregnant animal is altered compared to a control offspring. In one embodiment, the method includes administering an effective amount of a composition that includes active IGF-1 to a lactating animal, wherein a characteristic of an offspring nursed by the lactating animal is altered compared to a control offspring. In one embodiment, the characteristic of an offspring is selected from increased survival of offspring before weaning, increased weight of offspring at weaning, increased weight of offspring at end of growing phase, increased average daily gain, increased average daily food intake, and a combination thereof. In one embodiment, the increased weight of offspring at end of growing phase is selected from weight of living animal, hot carcass weight, and a combination thereof. The increased body weight of offspring at end of growing phase can be the result of increased bone density, increased bone growth, increased muscle growth, increased adipose tissue, increased head growth, increased organ growth, or a combination thereof. The increased muscle growth can be the result of increased number of muscle fibers, increased length of muscle fibers, or the combination thereof. The increased head growth can be due to increased brain growth. The increased organ growth can be heart, liver, lungs, stomach, intestines, or a combination thereof.

In one embodiment, the method includes administering an effective amount of a composition that includes active IGF-1 to a pregnant animal, wherein a characteristic of a litter of the pregnant animal is altered compared to a control litter. In one embodiment, the characteristic of a litter is increased number of offspring born alive, increased litter birth weight, increased offspring birth weight, reduced number of stillborn offspring, or a combination thereof. In one embodiment, when the animal is a pig the number of piglets having a weight of at least 2.5 pounds at birth is increased.

In one embodiment, the IGF-1 administered to the pregnant animal has been subjected to an activation process that increases the amount of active IGF-1. In one embodiment, the IGF-1 administered to the pregnant animal is obtained from a natural source that has been processed to increase the amount of active IGF-1. In one embodiment, the natural source is blood or a blood-derived product. In one embodiment, the natural source is milk or a milk-derived product. In one embodiment, the natural source is colostrum or a colostrum-derived product.

In one embodiment, the administering includes administering inactive IGF-1, wherein at least 20% of the total IGF-1 administered is active IGF-1. In one embodiment, the administering includes daily administration of at least 0.05 nanograms of active IGF-1 per kilogram bodyweight of the subject daily (ng/kg), at least 0.1 ng/kg, at least 0.5 ng/kg, at least 2 ng/kg, at least 5 ng/kg, at least 10 ng/kg, at least 20 ng/kg, at least 50 ng/kg, or at least 100 ng/kg. In one embodiment, the administering includes feeding the pregnant animal a food product comprising the active IGF-1. The food product can be administered throughout the pregnancy, during lactation following pregnancy, during estrus, before estrus, or a combination thereof.

In one embodiment, the subject is a bovine species, a porcine species, a cervid species, a canine species, a feline species, an equine species, a ovine species, an avian species, or a human.

The above summary of the application is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

Provided herein are methods for using a composition that includes one or more proteins. In one embodiment, a composition includes insulin-like growth factor (IGF), such as IGF-1 and/or IGF-2. IGF plays a role in regulation of normal physiology and a number of pathological states, including cancer, as well as cell proliferation and inhibition of cell death. IGF may affect different growth stages. Insulin-like growth factor 2 (IGF-2) is thought to be a primary growth factor required for early development while insulin-like growth factor 1 (IGF-1) expression is required for achieving maximal growth. Almost every cell in the human body is affected by IGF-1, especially cells in muscle, cartilage, bone, liver, kidney, nerves, skin, and lungs. IGF-1 can also regulate cell growth and development, especially in nerve cells, as well as DNA synthesis. IGFs are highly conserved between species, and the amino acid sequences of IGFs from different species are known and readily available to the skilled person.

Whether a protein is an IGF can be easily determined by the skilled person. For instance, polyclonal and monoclonal antibodies that specifically bind to IGF-1 and/or to IGF-2 are commercially available, and react with IGF from various species including human, equine, canine, bovine, porcine, and avian. These readily available antibodies lack cross-reactivity and/or interference by other closely related proteins and binding proteins. A single antibody or a panel of antibodies that recognizes different regions of an IGF, such as N-terminal, C-terminal, or amino acids present between the ends of the protein, may be used to determine whether a protein is an IGF protein. Methods for determining whether an IGF protein is active are known in the art and routine.

IGFs are proteins with high sequence similarity to insulin, but unlike insulin, IGFs associate with distinct binding proteins present in serum and other biological fluids (Baxter, 2000, Am J Physiol Endocrinol Metab, 278: E967-E976; Hwa et al., 1999, Endocrine Reviews, 20(6):761-787). Most IGF present in products derived from an animal, such as, but not limited to, blood and blood-derived products, milk and milk-derived products, and colostrum and colostrum-derived products, is bound to a binding protein. However, since these binding proteins inhibit the activity of IGF, most IGF present in animal derived products is inactive due to its being bound to a binding protein. For instance, less than 1% of IGF-1 in plasma is not bound to a binding protein (Carel et al., Safety of Recombinant Human Growth Hormone, In: Current Indications for Growth Hormone Therapy, 2nd rev. ed., vol. ed.: Hindmarsh, Karger, Switzerland, p. 48).

An IGF is considered to be active if it is not bound to a binding protein, and is considered to be inactive if it is bound to a binding protein. Active IGF is often referred in the art as free, unbound, bioactive, and/or active. Methods for measuring the concentration of active IGF are known to the skilled person and are routine. Assays, including solid phase sandwich ELISA assays, are commercially available that permit measurement of IGF that is not bound to a binding protein (e.g., R&D Systems, Minneapolis, Minn., catalog number DFG100).

A composition useful in the methods described herein includes active IGF, and optionally includes inactive IGF. In one embodiment, a composition is present in a food product. As used herein, a “food product” is a compound or mixture of compounds that can be ingested by a subject. A food product may be solid, semi-solid, or liquid. Examples include, but are not limited to, solid and semi-solid dairy products, including fermented dairy products, for instance yogurt. Beverages to which IGF can be added include milk, vegetable juice, fruit juice, soy milk, soybean milk, fermented soybean milk, and fruit flavored dairy beverages. In one embodiment, a food product is a feed for animal use, for instance, for feeding domesticated animals such as companion animals including, but not limited to, dogs or cats, and livestock including, but not limited to, bovine, porcine, avian, cervid, canine, feline, equine, or ovine animals. The appropriate concentration to add to a food product can be determined by the skilled person having knowledge of the level of active IGF in a composition and the approximate amount of food product to be eaten daily by the animal. In those embodiments where the animal is not a human, the skilled person will understand that estimating the amount of feed eaten by an animal can be based on the average for a population of animals.

In one embodiment, the composition may include a pharmaceutically acceptable carrier. As used herein “pharmaceutically acceptable carrier” includes, but is not limited to, saline, solvents, dispersion media, coatings, antibacterial and antifungal agents, isotonic and absorption delaying agents, and the like, compatible with pharmaceutical administration.

A composition compatible with pharmaceutical administration may be prepared by methods well known in the art of pharmacy. In general, a composition can be formulated to be compatible with its intended route of administration. A formulation may be solid or liquid. Administration may be systemic or local. In some aspects local administration may have advantages for site-specific, targeted disease management. Local administration may provide high, clinically effective concentrations directly to the treatment site, with less likelihood of causing systemic side effects.

Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. Appropriate dosage forms for enteral administration of a composition described herein include tablets, capsules or liquids, as well as a food product. Appropriate dosage forms for parenteral administration may include intravenous administration. Appropriate dosage forms for topical administration may include creams, ointments, and skin patch. Methods for making a pharmaceutically acceptable composition that includes IGF are known to the skilled person (Mahler et al., US Published Patent Application 20110152188).

Compositions can include sterile aqueous solutions or dispersions and sterile powders for the extemporaneous preparation of sterile solutions or dispersions. For intravenous administration, suitable carriers include physiological saline, bacteriostatic water, Cremophor EL™ (BASF, Parsippany, N.J.) or phosphate buffered saline (PBS). A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., the IGF, such as IGF-1) in the required amount in an appropriate solvent with one or a combination of ingredients, as required, followed by filtered sterilization. Generally, dispersions are prepared by incorporating the active compound into a sterile vehicle, which contains a basic dispersion medium and any other appropriate ingredients. In the case of sterile powders for the preparation of sterile injectable solutions, preferred methods of preparation include vacuum drying, spray-drying, and freeze-drying to yield a powder of the active ingredient plus any additional desired ingredient from a previously sterilized solution thereof.

A composition for use in topical administration may be formulated into many types of vehicles. Non-limiting examples of suitable vehicles include emulsions (e.g., oil-in-water, water-in-oil, silicone-in-water, water-in-silicone, water-in-oil-in-water, oil-in-water, oil-in-water-in-oil, oil-in-water-in-silicone, etc.), creams, lotions, solutions (both aqueous and hydro-alcoholic), anhydrous bases (such as lipsticks and powders), gels, ointments, or pastes (Williams, Transdermal and Topical Drug Delivery, Pharmaceutical Press, London, 2003). Variations and other vehicles will be apparent to the skilled artisan and are appropriate for use in the methods described herein.

It is also contemplated that an active compound may be encapsulated for delivery past the rumen of a ruminant or to a target area such as skin. Non-limiting examples of encapsulation techniques include the use of liposomes, vesicles, and/or nanoparticles (e.g., biodegradable and non-biodegradable colloidal particles comprising polymeric materials in which the ingredient is trapped, encapsulated, and/or absorbed, examples include nanospheres and nanocapsules) that can be used as delivery vehicles to deliver such ingredients to skin or digestive tract.

Oral compositions generally include an inert diluent or an edible carrier. In one embodiment, an oral composition includes a food product. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can also be prepared in the form of suppositories (e.g., with conventional suppository bases such as cocoa butter and other glycerides) or retention enemas for rectal delivery.

Pharmaceutical administration can be one or more times per day to one or more times per week, including once every other day. The skilled artisan will appreciate that certain factors may influence the dosage and timing required to effectively treat a subject, including but not limited to the severity of the infection, previous treatments, the general health and/or age of the subject, and other diseases present.

IGF useful in the methods described herein is obtainable from various sources. In one embodiment, a source is a natural source, such as a biological material from an animal. Examples of animals include, but are not limited to, vertebrates. Examples of vertebrates include, but are not limited to, mammals, such as a species that is bovine, porcine, cervid, canine, feline, equine, ovine, or a human. Another example of a vertebrate is an avian species. Examples of biological materials include, but are not limited to, blood and blood-derived products (e.g., whole blood, red blood cells, plasma, and derivatives thereof); milk and milk products (e.g., liquid milk, powdered milk, cheese, whey and whey products, curd, cheese, casein, lactose, milk fat, and derivatives thereof); colostrum and colostrum-derived products (e.g., liquid colostrum, dried colostrum); egg and egg-derived products (e.g., egg yolk, egg whites, egg membranes), bodily fluids (e.g., saliva, semen), and tissues (e.g., mucosa tissue, intestinal tissue, embryonic tissue). Examples of plasma include, but are not limited to, dried plasma and liquid plasma and fractions thereof, such as a lipid fraction. Examples of whey products include, but are not limited to liquid whey, whey protein concentrate, whey protein isolate, whey cream, whey retentate, procream, deproteinized whey, delactosed permeate. Examples of colostrum-derived products include, but are not limited to liquid colostrum whey, colostrum whey protein concentrate, colostrum whey protein, colostrum whey cream, colostrum whey retentate, colostrum procream, colostrum deproteinized whey, colostrum delactosed permeate, colostrum casein, colostrum lactose, colostrum curd. In one embodiment, the colostrum is colostrum secreted by a female within the first 6, the first 12, the first 24, or the first 48 hours after birth of offspring. In one embodiment, a natural source of IGF useful in the methods described herein is not colostrum. In one embodiment, IGF useful in the methods described herein is produced using recombinant techniques, or chemically or enzymatically synthesized. As used herein, IGF from a natural source, for instance, blood or a blood-derived product, is not produced using recombinant techniques, or chemically or enzymatically synthesized. Biological material, such as blood or a blood-derived product, useful for producing a composition with active IGF is readily available commercially.

A biological material may be enriched for the amount of total IGF present. A protein is enriched if it is present in a significantly higher fraction compared to the biological material from which the protein was enriched. The higher fraction may be, for instance, an increase of 2-fold, 4-fold, 6-fold, 10-fold, 100-fold, 1,000-fold, or 10,000-fold. Enrichment may result from reducing the amount of other molecules present in the biological material, e.g., proteins. However, the term enriched does not imply that there are no other molecules, e.g., proteins, present. Enriched simply means the relative amount of IGF has been significantly increased. The term “significant” indicates that the level of increase is useful to the person making such an increase. Enrichment of IGF is the result of intervention by a person to elevate the proportion of the protein.

Optionally, IGF can be purified from a biological material. A protein is considered to be purified if at least 75%, least 85%, or at least 95% of other components present in the biological material are removed. Proteins that are produced through chemical or recombinant means are considered to be purified. Methods for enriching and/or purifying IGF are known to the skilled person and are routine. Non-limiting examples of such procedures include fractionation on immunoaffinity or ion-exchange columns; ethanol precipitation; reverse phase HPLC; chromatography on silica or on an ion-exchange resin such as DEAE; chromatofocusing; SDS-PAGE; ammonium sulfate precipitation; gel filtration using, for example, cross-linked gels and/or hollow fiber; and ligand affinity chromatography.

Most, e.g., 95% to 99%, of the IGF obtained from many natural sources is associated with binding protein that causes the IGF to be inactive. Optionally, the amount of active IGF in a composition that is obtained from a natural source can be increased, e.g., the amount of total IGF in the composition may be unchanged but the amount of active IGF is increased, such that the amount of active IGF as a percentage of the total IGF is increased. Methods for increasing the amount of IGF that is active include processes routinely used to activate functional proteins obtained from a biological material. Such activation processes include, but are not limited to, exposing the biological material to heat shock, temperature adjustment, alcohol extraction, pH adjustment, enzyme addition, ionic changes, other chemical additions, and pressure, or combinations thereof (Daughaday et al., 1989, Endocr Rev. 10:68-91; Daughaday et al., 1987, J Lab Clin Med. 109:355-363; Breier et al., 1991, J Endocrinol. 128:347-357). Without intending to be limited by theory, such methods typically cause the dissociation of the binding protein from the IGF protein.

In one embodiment, the amount of active IGF in a composition that is obtained from a natural source can be increased by at least 2-fold, at least 4-fold, at least 5-fold, at least 10-fold, at least 20-fold, at least 50-fold, or at least 100-fold compared to the amount of active IGF in the composition before it is processed to activate IGF. In one embodiment, at least 20%, at least 30%, at least 40%, at least 50%, at least 60%, at least 70%, at least 80%, at least 90%, or 100% of the total IGF present is active. In one embodiment, no greater than 80%, no greater than 70%, no greater than 60%, no greater than 50%, no greater than 40%, no greater than 30%, no greater than 20%, no greater than 10%, or no greater than 5% of the total IGF present is inactive (e.g., bound to a binding protein). The composition subjected to the processing can be, for instance, a biological material from an animal, such as a blood or blood-derived product. Optionally, the biological material may be one that has been enriched for total IGF. Products made from natural sources and processed to activate IGF are commercially available as the product BETAGRO and IMMUTEIN (GBH Labs, Maple Grove, Minn.).

In those embodiments where the source of IGF is a natural source, the composition may typically include other components, including other proteins. Examples of other proteins that may be present include, but are not limited to, lysozymes, lactoferrin, growth factors, transfer factors, cytokines, and immunoglobulins.

Also provided are methods for using a composition described herein. In one embodiment, the method is for improving characteristics of offspring by administration of a composition to the mother of the offspring. In one embodiment, the method is for improving characteristics of a litter by administration of a composition to the mother of the litter. In one embodiment, the improvement is relative to a control, e.g., the improvement is determined by comparing offspring of a mother receiving a composition as described herein with offspring of a mother that did not receive a composition. The mother may be a pregnant female and/or a lactating female.

In one embodiment, the composition is administered to an animal that is pregnant. In one embodiment, the administration may begin at any time during the animal's pregnancy. In one embodiment, the administration begins the same day as pregnancy begins, no more than 2 days after pregnancy begins, no more than 5 days after pregnancy begins, no more than 10 days after pregnancy begins, or no more than 20 days after pregnancy begins. In one embodiment, the administration during pregnancy occurs during the time of certain developmental stages. Examples of developmental stages include the periods of gestation during which muscle development, mammary development, adipose development, or bone development occur. The timing of these stages varies depending upon the animal, but is known to the skilled person.

In one embodiment, the composition is administered to an animal before pregnancy, and continues into pregnancy. Administration before pregnancy may begin when the animal enters estrus, or before the animal enters estrus. In one embodiment, the composition is administered to an animal before pregnancy, and the administration stops during pregnancy. When the animal is a human the administration before pregnancy occurs when the human is attempting to become pregnant, for instance, when ovulation begins or during ovulation. When the animal is not a human, the administration before pregnancy occurs when the animal is on schedule to become pregnant. Whether the animal is a human or non-human, the administration may begin no more than 2 days before pregnancy, or no more than 5 days before pregnancy. In one embodiment, the administration begins when the animal enters estrus.

In one embodiment, the composition is administered to a lactating animal that is nursing offspring. In one embodiment, the composition is administered to a mother before pregnancy, or during pregnancy, and administration continues into lactation. In one embodiment, the administration may begin at any time during lactation, for instance, when lactation begins, no more than 2 days after lactation begins, no more than 5 days after lactation begins, no more than 10 days after lactation begins, or no more than 20 days after lactation begins. In one embodiment, the administration begins the same day that lactation begins. The composition may be administered to a lactating mother as long as it is nursing offspring.

Examples of animals include, but are not limited to, vertebrates. A vertebrate may be a monotocous species or a polytocous species. As used herein, “monotocous species” includes a species of animal that typically gives birth to a single offspring per pregnancy, including but not limited to, a bovine (such as a domesticated cow), an equine (such as a domesticated horse), an ovine, a caprine, a cervine (such as a deer), a human, and the like. Monotocous may include species that typically give birth to a single offspring but, occasionally give birth to two offspring during a single gestation period (i.e., “twins”). As used herein, “polytocous species” includes a species of animal that typically gives birth to multiple offspring (i.e., a “litter” of offspring) per pregnancy, such as, a porcine (such as a domesticated pig), a canine (such as a domesticated dog), a feline (such as a domesticated cat), and the like. In one embodiment, the animal is a female that has not given birth before, and in another embodiment the animal is a female that has given birth before. Another example of a vertebrate is an avian species (such as domesticated fowl).

In one embodiment, the characteristics of offspring, litter, or a combination thereof, that are improved include, but are not limited to, improvement of a characteristic at birth. Examples of improved characteristics at birth include, but are not limited to, increased number of offspring born alive, increased litter birth weight, increased offspring birth weight, reduced number of stillborn offspring, or a combination thereof. In one embodiment where the animal is a pig, the improvement includes increasing the number of piglets weighing at least 2.5 pounds at birth. In one embodiment, the improvement includes increased birth weight where the mother is a first-time mother.

In one embodiment, the characteristics of offspring that are improved include, but are not limited to, improvement of a characteristic after birth. Examples of improved characteristics after birth include, but are not limited to, increased survival of offspring before weaning, increased weight of offspring at weaning, or a combination thereof. In one embodiment, the method results in the mother having a reduced interval between weaning of offspring and onset of the next estrus cycle.

In one embodiment, the method improves characteristics of offspring after weaning and before adulthood. Such characteristics include, but are not limited to, increased average daily gain (ADG, average pounds gained by animal per day per period), increased average daily food intake (ADFI, average pounds consumed per animal per day), improved feed conversion ratio (ADFI/ADG, measurement of feed efficiency), or a combination thereof. In one embodiment, the increased average daily gain for an offspring born to an animal receiving the composition during pregnancy, lactation, or a combination thereof may be an increase of at least 0.5% of a control offspring, at least 1%, at least 3%, at least 5%, at least 7%, at least 9%, at least 11%, or at least 13%.

In one embodiment, the method results in improved weight gain. The weight gain may be at an interval of time after birth including during the offspring's nursing phase, during the phase between weaning and adulthood, and during adulthood. In one embodiment, the method results in increased weight at the time the animal is ready for market. The weight of an animal may be measured as body weight or hot carcass weight. Body weight refers to the weight of the living animal. Hot carcass weight is easily measured in a meat production animal, and refers to the weight of the unchilled carcass after hide, head, intestinal tract, and internal organs are removed. In one embodiment, the increase in body weight and hot carcass weight results from increased bone density, increased bone growth, increased muscle (e.g., increased number of muscle fibers, increased length of muscle fibers), increased adipose tissue, or a combination thereof. In one embodiment, the increase in body weight results from increased organ growth (e.g., internal organs, including, but not limited to, increased tissue of heart, liver, lungs, stomach, intestines), increased head growth (e.g., increased brain growth), increased skin growth, or a combination thereof The increase in weight of an offspring, when measured using body weight or hot carcass weight, may be an increase of at least 0.25% of a control offspring resulting from an animal not administered the composition, at least 0.5%, at least 1%, at least 2%, or at least 2.5%. For instance, when the offspring is a pig, the weight gain at market (either body weight or hot carcass weight) may be at least 0.5 pounds, at least 1, at least 3 pounds, or at least 4 pounds greater than a control.

In one embodiment, the administering can be feeding a composition that includes active IGF to the animal, for instance, pre-estrus, during estrus, during pregnancy, during lactation or a combination thereof. In one embodiment, active IGF can be present in a food product. The food product may naturally include the active IGF, or the food product may be supplemented with active IGF. In one embodiment, the addition of active IGF occurs by the supplementation of a food product with a biological material, such as a blood-derived product, e.g., plasma, that has been processed to increase the amount of active IGF. The amount of active IGF administered by feeding on a daily basis may be at least 0.05 ng/kg, at least 0.1 ng/kg, at least 0.5 ng/kg, at least 2 ng/kg, at least 5 ng/kg, at least 10 ng/kg, at least 20 ng/kg, at least 50 ng/kg, or at least 100 ng/kg, where ng refers to nanograms of active IGF and kg refers to kilograms bodyweight of the animal. In one embodiment, the amount of active IGF administered by feeding on a daily basis may be no greater than 150,000 ng /kg, no greater than 100,000 ng/kg, no greater than 50,000 ng/kg, or no greater than 20,000 ng/kg, where ng refers to nanograms of active IGF and kg refers to kilograms bodyweight of the animal. The active IGF administered may be active IGF-1, active IGF-2, or a combination thereof. In one embodiment, the active IGF administered is active IGF-1. In one embodiment there is no upper limit on the amount of active IGF administered.

The feed may be provided to an animal as part of its diet before pregnancy, throughout estrus or a portion thereof, throughout pregnancy or a portion thereof, throughout lactation of the offspring or a portion thereof, or a combination thereof In one embodiment, the feed may be provided to an animal for at least 1 day, at least 4 days, at least 7 days, at least 2 weeks, at least 3 weeks, at least 1 month, at least 2 months, or at least 3 months. In one embodiment, the feed may be provided to an animal for no greater than 24 months, no greater than 21 months, no greater than 18 months, no greater than 15 months, no greater than 12 months, no greater than 8 months, no greater than 5 months, no greater than 2 months, no greater than 3 weeks, or no greater than 2 weeks.

In one embodiment, the administering can be parenteral or topical. The amount of active IGF to be administered by a parenteral or topical route in the methods described herein can be determined by standard pharmaceutical procedures in cell cultures or experimental animals, e.g., for determining the ED₅₀ (the dose therapeutically effective in 50% of the population). The data obtained from cell culture assays and animal studies can be used in formulating a range of dosage for use in an animal. The dosage of active IGF lies preferably within a range that includes the ED₅₀ with little or no toxicity; however, it is expected that high levels of active IGF will not be detrimental to an animal. The dosage may vary within this range depending upon the dosage form employed and the route of administration utilized. For a compound used in the methods of the invention, the therapeutically effective dose can be estimated initially from cell culture assays and/or experimental animals.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLE 1

### Effect of a Plasma Product on Fetal Growth and Postnatal Development

### Materials and Methods

### Animals

Sows were divided into two groups. The control and treatment groups were 124 sows and 130 sows, respectively. The control group was fed a standard gestation diet, and the treatment group was fed the same standard gestation diet supplemented with a commercial plasma product (BETAGRO, GBH Labs, Maple Grove, Minn.) at 0.1%. After giving birth the control group was fed a standard lactation diet, and the treatment group was fed the same standard lactation diet supplemented with a commercial plasma product (BETAGRO, GBH Labs, Maple Grove, Minn.) at 0.1%.

At 24 to 36-hours postpartum, piglets were weighed individually and maintained within the appropriate group. Piglets were weighed at the beginning of each phase (see Table 1).

Mixed sex weanling piglets (1,240, 45% of the total pigs weaned from the sow phase of the trial) were housed on one nursery room containing 46 pens with 26-28 pigs/pen. Pigs were blocked by weaned date and weaning body weight (BW).

After the nursery phase 600 mixed sex weanling pigs were transferred in intact from their pens in the nursery to the finisher. The pigs were held in growing/finishing pens for 109 days.

Pigs were slaughtered using standard industry accepted practices. Live body weight was measured immediately before slaughter. After the hide, head, intestinal tract, and internal organs were removed the weight of the unchilled carcass of each animal was measured (hot carcass weight). Other measurements taken using the carcass were Fat-O-Meater (Carometic Food Technology A/S, Seoborg, Denmark) (to determine Fat depth, Loin depth, Yield, and % Carcass lean), and Meat Quality and Primal cuts (100 pigs/group). Loin measurements included Firmness score (1=soft to 5=firm), Yield % and pounds, Color Score (1 light and pale to 5 dark and red), Minolta L*, A*, and B*, Marbling, %, and Ultimate pH. Belly measurements included Yield % and pounds, Length Width and Depth, and Subjective firmness score (0.5=soft to 5=firm). Ham measurements included Yield % and pounds.

### Results

### Sows

There were improvements in the number of piglets born alive per litter (13.2 in the control group compared to 13.5 in the treatment group) and pre-weaning mortality (12.8% in the control group compared to 11.4% in the treatment group). In the treatment group there was a 4.3% increase in litter birth weight and a 2.1% increase in birth weight compared to the control group. These changes were not statistically significant but the trends were observed. There was a statistically significant decrease in stillborns in sows in the treatment group (a reduction from 5.37% to 3.57%, P=0.07), but no change in the occurrence of mummies.

Sows in the treatment group had a significantly reduced percentage of piglets weighing less than 2.5 pounds (33.3% of piglets in the control group were less than 2.5 pounds, while 29.6% of piglets in the treatment group were less than 2.5 pounds, P=0.05). When piglet birth weight was compared, there was an improvement in the treatment group when all litters were compared (a 2.1% increase from 2.89 pounds for the control group compared to 2.95 pounds for the treatment group). When the litters were divided by parity, gilts in the treatment group showed the largest increase (a 9.4% increase from 2.79 pounds to 3.05 pounds).

There were improvements in pre-weanling mortality (12.8% in control group and 11.4% in treatment group) and in the number of pigs weaned per litter (11.5 in control group and 11.9 in treatment group). These differences were not statistically significant but the trends were observed.

Sows in the treatment group weaned significantly heavier piglets (12.15 pounds compared to 12.68 pounds, P=0.005).

Sows in the treatment group had a significantly reduced wean-estrus interval (5.6 days compared to 4.52 days, P<0.05). The percentage of sows in the treatment group with a wean-estrus time of greater than 7 days was significantly reduced (8.1% compared to 3.1 percent, P=0.1).

### Nursery Stage

During the nursery phase piglets in the treatment group showed increases in average daily gain (the average pounds gained per day per period) and the average daily feed intake (average pounds consumed per animal per day) compared to the control group (Table 1). The body weight of piglets in the treatment group at the end of each phase was consistently higher than the piglets in the control group.

Growing/Finishing Stage

During the growing/finishing phase, the body weight of piglets in the treatment group at the end of each phase was consistently higher than the piglets in the control group (295.6 pounds compared to 290.1 pounds).

At the end of the growing/finishing stage pigs in the treatment group showed a significant increase in live body weight of 5.5 pounds (295.6 pounds in the treatment group compared to 290.1 in the control group, P=0.05) (Table 1), and a significant increase in hot carcass weight of 4.4 pounds (218.5 pounds in the treatment group compared to 214.1 pounds in the control group, P=0.07) (Table 2). From these data we infer that the increase in weight of pigs in the treatment group was due to an increase of 1.1 pounds in the head (e.g., brain), organs (e.g., internal organs), or both. In piglets from the treatment group there was a significant improvement in loin color, belly depth, and subjective flop score (Table 3).

TABLE 1
Effects in sow and nursery phase on pig growth performance
from weaning to harvest (values are least square means).

| | Control | Treatment | P-value |
|---|---|---|---|
| Nursery Phase | | | |
| No. Pens | 11 | 12 | |
| Pigs per pen | 26 | 26 | |
| Starting BW, lb | 13.02 | 12.80 | 0.12 |
| BW end of Phase 4, lb | 53.66 | 56.90 | 0.001 |
| Overall of Nursery Phase; 54-d | | | |
| ADG, lb | 0.75 | 0.82 | <0.001 |
| ADFI, lb | 1.10 | 1.20 | <0.001 |
| F:G | 1.47 | 1.46 | 0.95 |
| Growing-Finishing Phase | | | |
| No. Pens | 11 | 12 | |
| Starting BW, lb | 75.39 | 78.65 | 0.01 |
| BW end of Phase 8, lb | 290.1 | 295.6 | 0.05 |
| Overall of Growing-Finishing | | | |
| Phase; 109-d | | | |
| ADG, lb | 2.04 | 2.07 | 0.53 |
| ADFI, lb | 5.48 | 5.65 | 0.06 |
| F:G | 2.69 | 2.73 | 0.17 |
| Removal, % | | | |
| Nursery Phase | 1.85 | 1.23 | |
| Growing-Finishing Phase | 3.45 | 2.83 | |

BW, Body weight;
ADG, Average daily gain (average pounds gained per day period);
ADFI, Average daily feed intake (average pounds consumed per animal per day);
F:G, feed to gain, also know as “feed conversion ratio” (measurement of feed efficiency, which equals amount of ADFI/ADG);
removal %, percentage of animals removed due to death, poor health or injury.

TABLE 2
Effects of feeding supplement in sow phase on carcass
characteristics (values are least square means).

| | Control | Treatment | P-value |
|---|---|---|---|
| No. Pens | 11 | 12 | |
| Live BW, lb | 290.1 | 295.6 | 0.05 |
| CV,¹ % | 8.63 | 9.30 | |
| Live BW <290 lb, % | 50.0 | 45.6 | |
| HCW, lb | 214.1 | 218.5 | 0.07 |
| CV,² % | 9.32 | 9.42 | |
| HCW <220 lb, % | 62.1 | 50.7 | |
| Carcass Yield, % | 73.76 | 73.92 | 0.54 |
| Fat Depth, mm | 17.43 | 17.49 | 0.74 |
| Loin Depth, mm | 63.00 | 64.45 | 0.27 |
| Carcass Lean, % | 55.78 | 55.93 | 0.99 |

¹Coefficient of variation (CV) of live BW was calculated using each individual pig data from four pens per treatment (Control = 104 pigs, Treatment = 103 pigs)
²Coefficient of variation (CV) of HCW was calculated using each individual pig data from 11 or 12 pens per treatment (Control = 272 pigs; Treatment = 288 pigs)
BW, body weight;
HCW, hot carcass weight.

TABLE 3
Effects of feeding supplement in sow phase on quality of
primal cuts (values are least square means).

| | Control | Treatment | P-value |
|---|---|---|---|
| Boneless Loin | | | |
| No. Pigs¹ | 106 | 106 | |
| Weight, lb | 8.10 | 8.26 | 0.61 |
| CV, % | 11.6 | 11.0 | |
| Yield, % of HCW | 3.74 | 3.83 | 0.77 |
| Color Score - primary² | 3.20 | 3.24 | 0.01 |
| Color Score - secondary² | 3.16 | 3.22 | 0.01 |
| Marbling, % | 1.79 | 1.71 | 0.45 |
| Firmness Score³ | 2.69 | 2.64 | 0.65 |
| Ultimate pH | 5.65 | 5.68 | 0.26 |
| Minolta L* | 44.97 | 44.51 | 0.08 |
| Minolta a* | 7.96 | 8.22 | 0.01 |
| Minolta b* | 0.06 | 0.25 | 0.14 |
| Primal Ham | | | |
| No. Pigs¹ | 107 | 107 | |
| Weight, lb | 25.7 | 25.9 | 0.69 |
| CV, % | 9.3 | 7.5 | |
| Yield, % of HCW | 11.9 | 12.0 | 0.35 |
| Belly | | | |
| No. Pigs¹ | 105 | 108 | |
| Weight, lb | 18.81 | 18.39 | 0.91 |
| CV, % | 14.8 | 14.8 | |
| Yield, % of HCW | 8.65 | 8.47 | 0.82 |
| Length, inch | 28.67 | 28.66 | 0.94 |
| Width, inch | 14.35 | 14.29 | 0.72 |
| Depth, inch | 1.03 | 0.98 | 0.10 |
| Subjective Flop Score⁴ | 2.67 | 2.54 | 0.07 |

¹Each individual pig from four pens per treatment were selected for evaluation of primal cuts
²Loin color score: 1 - light and pale; 5 - dark and red (NPPC, 2000)
³Loin firmness score: 1 - soft; 5 = firm (NPPC, 2000)
⁴Belly subjective flop score: 0.5 = soft; 5 = firm

### EXAMPLE 2

In a second trial at a commercial research swine operation, 200 sows were divided into two groups of 96 (control group) and 104 (treatment group), respectively. The control group was fed a standard gestation diet, and the treatment group was fed the same standard gestation diet supplemented with a commercial plasma product (BETAGRO, GBH Labs, Maple Grove, Minn.). After giving birth the control group was fed a standard lactation diet, and the treatment group was fed the same standard lactation diet supplemented with a commercial plasma product (BETAGRO, GBH Labs, Maple Grove, Minn.) at 2 pounds per ton of standard lactation diet (lbs/ton) then during the nursery with 6 lbs/ton in Phase 1, at 3 lbs/ton during Phase 2, and no supplement in Phase 3 and 4. Piglets were weighed, maintained, transferred from nursery pens to finisher, and slaughtered as described in Example 1.

### Results

### Sows

There were improvements in the number of piglets born alive per litter (13.3 in the control group compared to 13.6 in the treatment group) and pre-weaning mortality (9.3% in the control group compared to 8.78% in the treatment group). There was a statistically significant decrease in the occurrence of mummies (a reduction from 6.02% to 3.8%, P=0.1), and a slight increase in the number of stillborns.

Sows in the treatment group weaned significantly heavier piglets (10.99 pounds compared to 11.57 pounds, P=0.002), produced significantly heavier piglets through the nursery phase (P=0.002), and these piglets ate significantly more feed (P=0.03).

Sows in the treatment group had a significantly reduced wean-estrus interval (4.83 days compared to 4.51 days, P<0.007). The percentage of sows in the treatment group with a wean-estrus time of greater than 7 days was reduced (3.13% compared to 1.92 percent).

### Nursery Stage

During the nursery phase piglets in the treatment group showed increases in average daily feed intake (average pounds consumed per animal per day) of 1.32 for the control group compared to 1.39 for the treatment group (P=0.30). The body weight of piglets in the treatment group at the end of the nursery phase was higher than the control group (59.5 pounds for the treatment group and 56.46 pounds for the control group, P=0.60). These changes were not statistically significant but the trends were observed.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for altering a characteristic of an offspring, the method comprising:
administering an effective amount of a composition to a pregnant animal wherein the composition comprises active IGF-1, wherein a characteristic of an offspring of the pregnant animal is altered compared to a control offspring.

2. (canceled)

3. The method of claim 2 wherein the increased weight of offspring at end of growing phase is selected from weight of living animal, hot carcass weight, and a combination thereof.

4. The method of claim 2 wherein the increased body weight of offspring at end of growing phase is the result of increased bone density, increased bone growth, increased muscle growth, increased adipose tissue, increased head growth, increased organ growth, or a combination thereof.

5. The method of claim 4 wherein the increased muscle growth is selected from increased number of muscle fibers, increased length of muscle fibers, and the combination thereof.

6. The method of claim 4 wherein the increased head growth comprises increased brain growth.

7. The method of claim 4 wherein the increased organ growth is selected from heart, liver, lungs, stomach, intestines, and a combination thereof.

8. The method of claim 1 wherein the IGF-1 administered to the pregnant animal has been subjected to an activation process that increases the amount of active IGF-1.

9. The method of claim 1 wherein the IGF-1 administered to the pregnant animal is obtained from a natural source that has been processed to increase the amount of active IGF-1.

10. The method of claim 1 wherein the administering further comprises administering inactive IGF-1, wherein at least 20% of the total IGF-1 administered is active IGF-1.

11. The method of claim 9 wherein the natural source is blood or a blood-derived product.

12. The method of claim 9 wherein the natural source is milk or a milk-derived product.

13. The method of claim 9 wherein the natural source is colostrum or a colostrum-derived product.

14. (canceled)

15. The method of claim 1 wherein the administering comprises feeding the pregnant animal a food product comprising the active IGF-1.

16. The method of claim 15 wherein the food product is administered throughout the pregnancy.

17. The method of claim 15 further comprising administering the food product during lactation following pregnancy.

18. The method of claim 15 further comprising administering the food product during estrus.

19. The method of claim 15 further comprising administering the food product before estrus.

20. (canceled)

21. A method for altering a characteristic of a litter, the method comprising:
administering an effective amount of a composition to a pregnant animal wherein the composition comprises active IGF-1, wherein a characteristic of a litter of the pregnant animal is altered compared to a control litter.

22. (canceled)

23. The method of claim 22 wherein the animal is a pig, wherein the number of piglets having a weight of at least 2.5 pounds at birth is increased.

24.-36. (canceled)

37. A method for altering a characteristic of an offspring, the method comprising:
administering an effective amount of a composition to a lactating animal wherein the composition comprises active IGF-1, wherein a characteristic of an offspring nursed by the lactating animal is altered compared to a control offspring nursed by a lactating animal that is not administered the composition.

38.-56. (canceled)
